(19) United States
(12) Patent Application Publication
(10) Pub. No.: US 2006/0085873 A1
(43) Pub. Date: Apr. 20, 2006

(54) Plasmid vector for expression of hepatitis B virus genes in plants, transgenic cell lines for the genes, and the use thereof in manufacture

(76) Inventors: Jun Sheng, Changchun City (CN); Dan Liu, Changchun City (CN); Xiaoyu Liu, Changchun City (CN); Qiao Guo, Changchun City (CN); Haipeng Yu, Changchun City (CN); Juan Li, Changchun City (CN); Yuejun Hui, Changchun City (CN); Zhiwu Wang, Changchun City (CN); Xuemei Zhang, Changchun City (CN)

Correspondence Address: HASSE & NESBITT LLC, 7550 CENTRAL PARK BLVD., MASON, OH, 45040, US

(21) Appl. No.: 11/235,569

(22) Filed: Sep. 26, 2005

Related U.S. Application Data
(63) Continuation-in-part of application No. PCT/CN04/00254, filed on Mar. 25, 2004.

(30) Foreign Application Priority Data
Mar. 26, 2003 (CN) CN 03120981.5

(51) Int. Cl. A01H 1/00; C12N 1/20; C12N 5/04

(52) U.S. Cl. 800/288; 800/294; 435/252.2; 435/419

(57) ABSTRACT

Transgenic plant cell lines and the plant cell expression systems which express HBsAg. The present invention further relates to the method of preparing the above-mentioned cell lines or expression systems, and the use of the above-mentioned cell lines or expression systems in the manufacture of HBsAg protein. The cell lines or expression systems disclosed in the invention can be used to prepare vaccine against hepatitis B virus.

### CROSS REFERENCE TO RELATED APPLICATIONS

This is a continuation-in-part of International Application PCT/CN2004/000254, with an international filing date of Mar. 25, 2004.

### TECHNOLOGICAL FIELD

The present invention relates to the field of transgenic technology of plants. More specifically, the invention relates to constructing cell lines of transgenic ginseng callus which express the surface antigen of hepatitis B virus (HBsAg), obtaining the HBsAg protein, and applying the cell lines in the manufacture of vaccine against hepatitis B virus.

### BACKGROUND OF THE INVENTION

Hepatitis B is currently one of the most prevalent and most harmful infectious diseases in China. At present there are about 350 million carriers of hepatitis B in the world, over 120 million of those are in China. Thirty million people who are currently suffer from chronic hepatitis B are on the move in the society, and each year over two million cases of acute hepatitis take place. About 350 thousand people die of liver disease each year, half of those die of primary liver cancer. Hepatitis B has greatly affected the social stability of China and the development of the national economy.

The prevention of hepatitis B virus infection will still depend on the effective function of vaccines. Widely inoculation against hepatitis B is quite necessary in China, and there exists extremely large demands on hepatitis B vaccines. However, since the expression level of the expression systems (for CHO— or yeast-vaccine) is low, the production capacity now available in our country is therefore restricted, not being able to satisfy the social demands at all (the vacancy in the market each year is 30 million pieces). It is quite urgent to find a brand new expression system.

With the development of plant tissue and cell culture techniques, especially the ones of plant cell suspension culture, the technical plateau of cultivating plant tissue and cells through large-scale fermentation has shown a vast range of prospect for development.

Large-scale production of new type genetically engineered hepatitis B vaccine through industrialized fermentation and culture of ginseng callus cells has shown a tremendous technological advantage in many aspects, including the substantial increase in the expression level, the simplification of the process of extraction and purification, the development of brand new excellent formulation, the safety of the product, and the decrease of the production cost. As biological reactors, transgenic plant tissues and cells have a vast prospect of application. The present invention is the first to make a breakthrough in this field, and thus provides a technological plateau for the expression and production of medical recombinant proteins by means of culturing ginseng callus, and such a plateau is unique to China.

### SUMMARY OF THE INVENTION

The present invention relates to a gene construct comprising the HBsAg gene, wherein a promoter which enables the highly-efficient expression of the said HBsAg gene in plant cells is assembled at the 5′ end of the gene, and a terminator which enhances the expression of the said HBsAg gene is assembled at the 3′ end of the gene. Preferably the said promoter is CaMV 35S promoter. And preferably the said terminator is nos terminator. The gene construct according to the present invention can be used in the expression of HBsAg in plant cells.

The present invention further relates to the transgenic plant cell lines comprising the above-mentioned gene construct. Especially, the invention relates to transgenic ginseng cell lines, and preferably the said ginseng cells are derived from the ginseng callus. In a preferred embodiment, the transgenic plant cell lines according to the invention further contains a selective marker, which is preferably NPT II. Such a marker can be used in the selection with kanamycin in prokaryotic cells, and be used in the selection with kanamycin, neomycin and G418 in eukaryotic cells.

The present invention further relates to the method of preparing the transgenic plant cell lines according to the invention, which comprises:
1) Introducing into plant cells the construct or vector according to the invention comprising the HBsAg gene, and
2) Enabling the said plant cells to express HBsAg protein.

In one embodiment, the HBsAg gene was introduced into the plant cells by vectors comprising the HBsAg gene. In such a vector, a promoter which enables the highly-efficient expression of the HBsAg gene in plant cells was assembled at the 5′ end of the gene, and the promoter was preferably a CaMV 35S promoter. And a terminator which enhances the expression of the said HBsAg gene was assembled at the 3′ end of the gene, and the terminator was preferably a nos terminator. The said vector was preferably pBIBSa or pBIBSa.

The construct or vector according to the invention can be introduced into the plant cells by means such as Agrobacterium tumefaciens infection, gene gun method, pollen introduction, virus-mediated method, PEG-mediated method, induction through electric shock, microinjection, laser transformation, ultrasonic transformation, and liposome transformation. Preferably, in the method of preparing the transgenic plant cell lines according to the invention, the plant cells are infected by Agrobacterium tumefaciens which carries the HBsAg gene, so that the HBsAg gene can be introduced into the plant cells. More preferably, the said HBsAg gene is introduced into cells of Agrobacterium tumefaciens by the above-mentioned construct or vector, and then the cells of Agrobacterium tumefaciens are used to infect the plant cells, so that the gene can be introduced into the plant cells. In one embodiment of the invention, the said Agrobacterium tumefaciens infection was performed by co-culturing the suspended plant cells and Agrobacterium tumefaciens. During the Agrobacterium tumefaciens infection, phenolic compounds can be used to induce the activation of genes in the Vir region of Agrobacterium tumefaciens, so as to enhance the transformation efficiency of Agrobacterium tumefaciens. The said phenolic compounds are selected from catechol, gallic acid, pyrogallic acid, protocatechuic acid, vanillin, acetosyringone, and hydroxyl acetosyringone, and is most preferably acetosyringone (AS).

The transgenic plant cell lines according to the invention can be used in the manufacuture of the HBsAg protein, and thereafter of hepatitis B vaccine.

The present invention further relates to a plant cell expression system which expresses the HBsAg protein, wherein the said system contains a construct comprising the HBsAg gene, a promoter which enables the highly-efficient expression of the gene in plant cells, and a terminator which enhances the expression of the gene. Preferably the promoter is CaMV 35S promoter, and preferably the terminator is nos terminator. The expression system according to the invention can further comprise a selective marker, which is preferably NPT II, and such a marker can be used in the selection with kanamycin in prokaryotic cells, and in the selection in eukaryotic cells with kanamycin, neomycin and G418. The marker can also be comprised in the construct according to the invention. Preferably the plant expression system according to the invention is the one from ginseng, most preferably the one derived from the callus of ginseng.

The present invention further relates to the method of preparing the above-mentioned expression system, which comprises:
1) introducing into the said expression system the construct according to the invention which comprises the HBsAg gene, a promoter which enables the highly-efficient expression of the gene in plant cells, and a terminator which enhances the expression of the gene; and
2) enabling the expression system to express the HBsAg protein;
   wherein the said promoter is preferably CaMV 35S promoter, and the said terminator is preferably nos terminator.

In one of the embodiments of the present invention, the said construct was comprised in a vector, which is preferably pBIBSa or pBIBSa.

The above-mentioned construct or vector can be introduced into plant cells by means such as Agrobacterium tumefaciens infection, gene gun method, pollen introduction, virus-mediated method, PEG-mediated method, induction through electric shock, microinjection, laser transformation, ultrasonic transformation, and liposome transformation. And infection through Agrobacterium tumefaciens is preferred.

Alternatively, the above-mentioned construct according to the invention can be directly introduced into Agrobacterium tumefaciens, and then be delivered into the said expression system through Agrobacterium tumefaciens.

Whether through vectors, or by the means of being directly introduced into Agrobacterium tumefaciens, the construct according to the invention can be introduced into plant cells by co-culturing the suspended plant cells and Agrobacterium tumefaciens. During such a process, phenolic compounds can be used to induce the activation of genes in the Vir region of Agrobacterium tumefaciens, so as to enhance the transformation efficiency of Agrobacterium tumefaciens. Said phenolic compounds are selected from catechol, gallic acid, pyrogallic acid, protocatechuic acid, vanillin, acetosyringone, and hydroxyl acetosyringone, and is preferably acetosyringone (AS).

The above-mentioned expression system of the present invention is preferably derived from ginseng, more preferably from the callus of ginseng. Such an expression system can be used in the manufacture of the HBsAg protein, and thereafter of hepatitis B vaccine.

Accordingly, the present invention relates to the construction of the vector plasmid for expression in plant cell, the enhancement of the efficiency of gene transformation, the selection and establishment of transgenic cell lines, and the development and application thereof.

Successful gene transformation depends on the establishment of a favorable plant acceptor system. Specific conditions are as follows:
1. The acceptor cells used for plant gene transformation must be prone to be regenerated with high frequency, stability and reproducibility.
2. The plant acceptor system should have relatively high level of genetic stability. After it receives foreign DNAs, its division and differentiation shall not be influenced, and the foreign genes can be stably passed down to the progenies, so that the genetic stability can be maintained.
3. A steady supply of acceptor cells is also necessary for a high-yield tissue culture regeneration system to be established and applied to gene transformation. Because the frequency of plant gene transformation is very low and many experiments are needed to obtain a successful gene transformation, the acceptor cells should be easy to be obtained, and should also be able to be provided in large amount.
4. The antibiotic-resistance is usually used in selection of transformants, therefore those non-transformed cells that are sensitive to antibiotics should be chosen as acceptor cells.
5. Agrobacterium tumefaciens can be used as vectors to mediate the gene transformation of plants, yet the use of such a method is restricted with the range of hosts that can be chosen. Different plants, and even different tissues and cells of the same plant have different sensitivity to Agrobacterium tumefaciens invasion, therefore the sensitivity of the acceptor system to Agrobacterium tumefaciens invasion must be tested before any Agrobacterium tumefaciens transformation system is chosen.

Furthermore, when establishing an acceptor system for the gene transformation of plants, care must be taken whether the intended system has economic value, or has potential value for production application. For this reason, in one of the embodiments of the present invention, the plant cell expression vectors for HBsAg, and the HBsAg transgenic ginseng callus cell lines that had been transformed with the said vector were constructed.

In the present invention, the HBsAg gene was used to replace the GUS gene in the plant cell expression vector, the plasmid pBI121 (NCBl Accession Number AF485783; Chen, P. Y., Wang, C. K., Soong, S. C. and To, K. Y.: Complete sequence of the binary vector pBI121 and its application in cloning T-DNA insertion from transgenic plants. Mol. Breed. 2003, 11, 287-293) in order to construct the vector of plasmid pBIBS. The vector was then introduced into Agrobacterium tumefaciens LBA4404 competent cells, and the Agrobacterium tumefaciens was used to infect ginseng callus, so that the vector pBIBS plasmid was introduced into the plant cells. Resistant cell lines were obtained on G418 selective media by the expression of the NPTII gene. Chromosomes of the resistant cell lines were extracted to identify the integration of the HBsAg gene. Proteins of the resistant cell lines were also extracted to identify the expression of HBsAg.

Between the 5′ end of the GUS gene and the CaMV 35S promoter of the pBI121 plasmid, three restriction sites are available, namely Xbal, BamHI and SamI. Between the 3′ end of the GUS gene and the nos terminator, one Sstl restriction site is available. The sequence of the HBsAg gene contains XbaI and BamHI sites, therefore during the construction of pBIBS, one can only choose the 5′ SmaI site and the 3′ Sstl site. Since the base sequence of SmaI restriction site is CCCGGG, too high a GC content might influence the specificity of PCR amplification. This problem can be solved by using the characteristics of SmaI which has blunt ends in its restriction site. When designing the 5′ end primer of the invention, other restriction sites were used to replace the SmaI site. Restriction sites with blunt end can be introduced directly to be ligated with the fragment that has been digested with SmaI; one can also introduce a site with sticky end, turn it into a blunt end by mung-bean nuclease or T4 DNA polymerase, and then ligate it with the fragment that has been digested with SmaI; one can also introduce a restriction site with sticky end, and afterwards introduce such a fragment into an intermediate vector, using the blunt ends that are on the intermediate vector to perform ligation reaction.

The plasmid pBIBS has reserved the NPT II (neomycin phosphotransferase) gene of the plasmid pBI121. The NPT II gene is the most widely used selective marker in plant gene transformation. The product encoded by the gene has resistance against aminoglycoside antibiotics such as kanamycin, neomycin and G418. Some prokaryotic cells and some eukaryotic ones are more sensitive to kanamycin, while neomycin and G418 works only on eukaryotes. With such a property of the NPT II gene product, the plasmid pBIBS can be widely used in the selection of Escherichia coli, Agrobacterium tumefaciens, an plant cells.

E. coli XL-I blue (Bullock, W. O., J. M. Fernandez, and J. M. Short, 1987, XL1-Blue, A high efficiency plasmid transforming recA Escherichia coli strain with beta-galactosidase selection, BioTechniques 5:376) does not have resistance against kanamycin. Kanamycin resistance can be endowed after the cells are transformed by the vector pBIBS, therefore can be used in the construction of vectors to select for resistant clones. Agrobacterium tumefaciens LBA4404 does not have resistance against kanamycin. Kanamycin resistance can be endowed to the cell after it is transformed by the vector of pBIBS, so that it can grow on the media carrying kanamycin. Agrobacterium tumefaciens cells carrying the plasmid with resistance can hence be selected. Ginseng callus do not have resistance against kanamycin or G418. After they are infected by the Agrobacterium tumefaciens which carry the plasmid with resistance, they can grow on the media carrying kanamycin or G418. Transgenic ginseng callus cells can hence be selected.

Some common phenolic compounds in plants can be used to induce the activation of genes in the Vir region of Agrobacterium tumefaciens, so as to enhance the transformation efficiency of Agrobacterium tumefaciens. These compounds include catechol, gallic acid, pyrogallic acid, protocatechuic acid, vanillin, acetosyringone (AS), and hydroxyl acetosyringone. And acetosyringone has the best effect in induction. During the process of pre-culturing Agrobacterium tumefaciens and its co-culturing with plant cells according to the invention, acetosyringone is added to enhance the transformation efficiency of Agrobacterium tumefaciens.

In one of the embodiments, the plant acceptor for gene transformation according to the invention was a ginseng callus cell line. This ginseng callus cell line is a continuous cell line that has been kept for 28 years of stable growth, and it can be passed on to expand the scale of culture, so that enough cells can be produced for gene transformation. Having reversed from the differentiated cells to the dedifferentiated meristematic cells, callus is prone to accept foreign genes, therefore can be used enhance the transformation efficiency. Non-transformed ginseng callus cells are sensitive to both kanamycin and G418, while G418 has a more obvious inhibitive effect. Therefore G418 can be used as a selective marker for transgenic cells.

According to the method of the invention, the vectors pBIBSa and pBIBSb carrying HBsAg gene for expression in plant cell were obtained, and the HBsAg transgenic ginseng callus cell lines were further obtained.

### BRIEF DESCRIPTION OF THE DRAWINGS

FIG. 1 is a schematic diagram of the construction of plasmid pMDBS.

FIG. 2 is a schematic diagram of the construction of plasmid pSKBS.

FIG. 3 is a schematic diagram of the construction of plasmid pBIBSa.

FIG. 4 is a schematic diagram of the construction of plasmid pBIBSb.

### DETAILED EMBODIMENTS OF THE INVENTION

### EXAMPLES

### Example 1

Construction of the Plant Cell Expression Vector for the HBsAg Gene

1. Extracting Chromosomal DNA From C28 Cells

C28 cells (comprising the HBsAg S gene, provided by the Changchun Institute of Biological Products, Xi'an boulevard 137, Changchun, Ji Lin Province, 130062) that were at least 80% confluent were taken. The cells were washed with PBS buffer, digested with 25% trypsin, and resuspended in TBS buffer. The cells were harvested by centrifugation. The cells thus obtained were then resuspended in TE buffer (pH 8.0), so that the concentration is 5×10⁷/ml. 1 ml of the suspension is added to a 10 ml extraction buffer (10 mmol/L Tris.Cl (pH 8.0), 0.1 mol/L EDTA (pH 8.0), 20 μg/ml pancrease RNAase, 0.5SDS), and the suspension was incubated at 37° C. for 1 hour. Proteinase K was added till a final concentration of 100 μg/ml, and cells were incubated at 50° C. for 3 hours. Cells were cooled to room temperature, and equal volumn of balanced phenol was added to extract the DNA. The water phase was collected by centrifugation, and two volumns of water-free ethanol were added. After mixing, the mixture was centrifuged, and the supernatant were abandoned. The DNA pellet was washed with 75% ethanol, centrifuged, and the supernatant was abandoned. The DNA pellet was then dried and collected. The DNA was resolved in 500 μl of TE (pH 8.0), and kept in 4° C.

2. Obtaining the HBsAg Gene

A pair of primers were designed as follows:

P1: 5′-ACTCGAGACATGGAGAACACAG-3′;

P2: 5′-ACGTCGAGCTCAAATGTATAC-3′.

PCR amplification was performed using the chromosomal DNA of C28 cells as template. An amplification fragment of 700 bp with the XhoI site and ATG start codon upstream and the Sst I site and TGA stop codon downstream was obtained.

3. Constructing the Cloning Vector

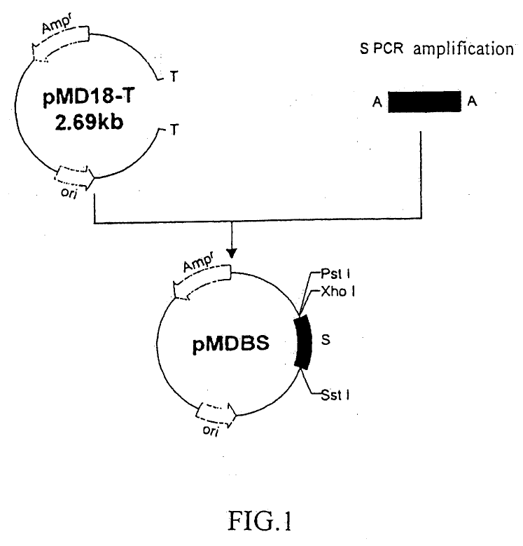

a. Ligation reaction was performed using the base As that were extrusive at both ends of the PCR amplification fragment and the base Ts that were extrusive at both ends of the pMD18-T vector (commercially available from Bao Biotechnology (Da Lian) Ltd., Second Northeast Street 19, Opening Region for Economy and Technology, Da Lian, Liaoning Province, 116600). Cloning plasmid pMDBS (FIG. 1) was obtained, which carried the Hind III, Sph I, Pst I, Sal I, and Xho I sites upstream of the S gene, and Sst I, Xba I, Bam HI, Sma I, Kpn I, Sac I, and Eco RI sites downstream of the S gene.

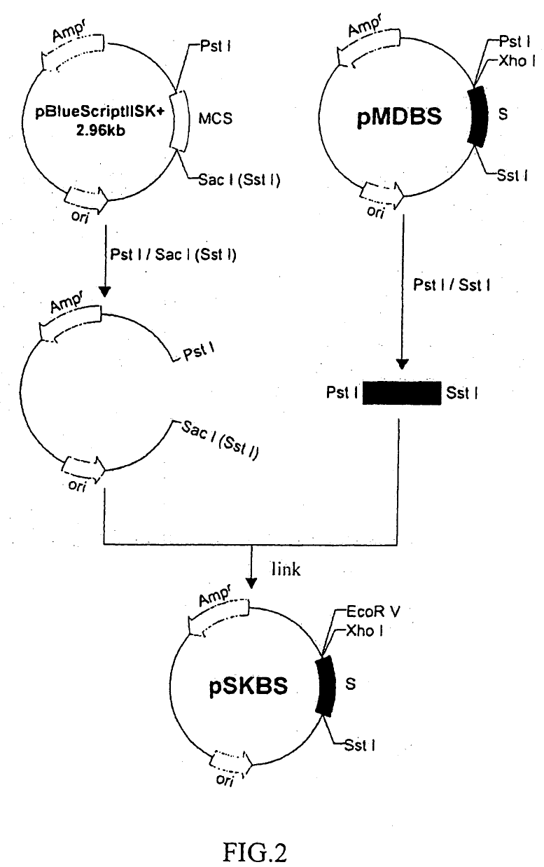

b. The plasmids pMDBS and pBluescript II SK+ (Shor, J. M., J. M. Fernandez, J. A. Sorge, and W. D. Huse, 1988, λZAP: A bacteriophage λ expression vector with in vivo excision properties, Nucleic Acids Res. 16: 7583) were digested with Pst I and Sst I, and the fragments of the S gene and the pBluescript 11 SK+ vector were recovered from agarose gel. The fragments were ligated to obtain the cloning plasmid pSKBS (FIG. 2), which carried the Kpn I, Apa I, Xho I, Sal I, Hind III, Eco RV, Eco RI and Pst I sites upstream of the S gene, and the Sst I site downstream of it.

4. Two Ways to Construct the Expression Vector

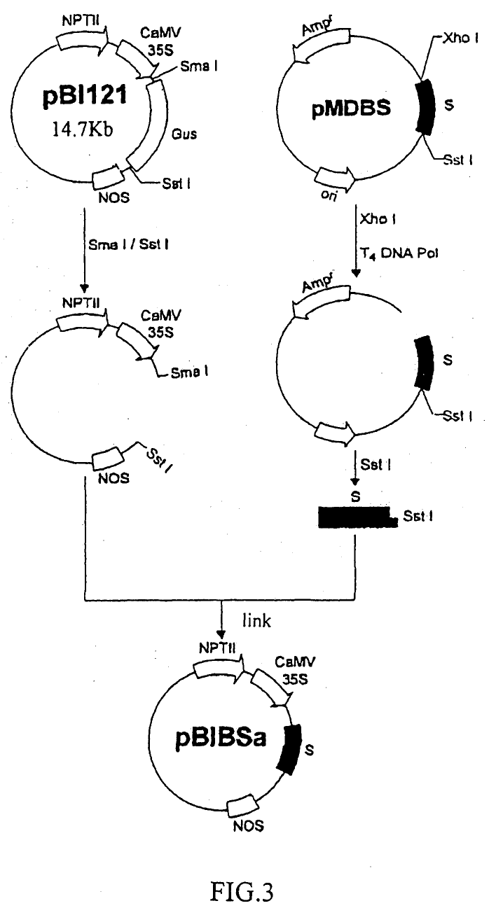

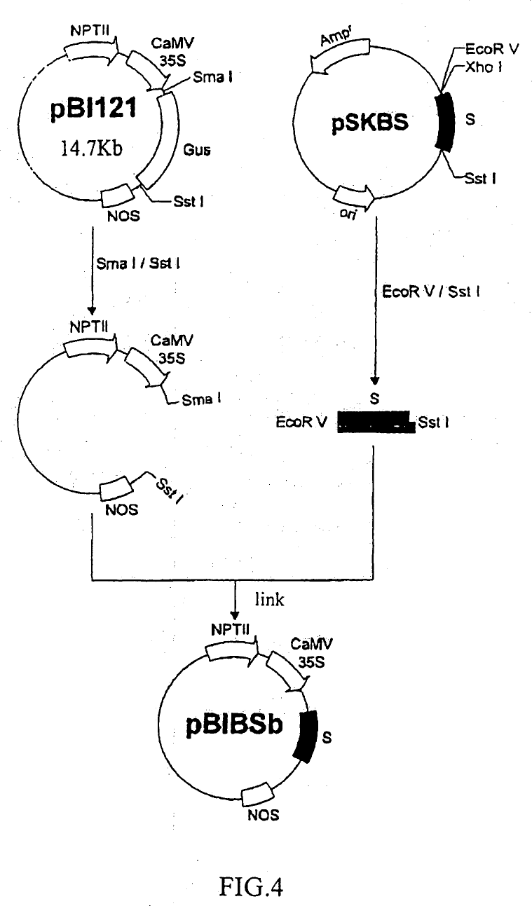

The plasmid pBI121 was digested with Sma I and Sst I, and the fragment of the vector pBI121 was recovered from agarose gel.
a. The plasmid pMDBS was digested with Xho I. The fragment obtained was recovered from agarose gel, and the sticky ends were made blunt by T4 DNA polymerase. The fragment obtained was then digested with Sst I, and fragment of the S gene was recovered from agarose gel. Such a fragment was then ligated with fragment from the vector pBI121 to obtain the plasmid vector pBIBSa comprising the hepatitis B gene for plant cell expression (FIG. 3).
b. The plasmid pSKBS was digested with Eco RV and Sst I, and fragment of the S gene was recovered from agarose gel. Such a fragment was then ligated with fragment from the vector pBI121 to obtain the plasmid vector pBIBSb comprising the HBsAg gene for plant cell expression (FIG. 4).

### Example 2

Transformation of A. tumefaciens with Expression Vectors

1. Preparing Competent Cells of Agrobacterium tumefaciens LBA4404

Single colony of Agrobacterium tumefaciens LBA4404 (Hoekema, A., P. R. Hirsh, P. J. J. Hooykaas, and R. A. Schilperoort, 1983, A binary plant vector strategy based on separation of the Vir- and T-region of the Agrobacterium tumefaciens Ti plasmid. Nature (London) 303: 107-118; Ooms, G., P. J. J. Hooykaas, R. J. M. Van Veen, P. Van Beelen, A. J. G. Regensburg-Tuink, and R. A. Schillperoort, 1982. Octopine Ti plasmid deletion mutants of Agrobacterium tumefaciens with emphasis on the right side of the T-region.Plasmid 7:15-29; Jen G C, Chilton M D. Activity of T-DNA borders in plant cell transformation by mini-T plasmids, J Bacteriol, 1986 May; 166 (2): 491-9) was inoculated in 5 ml of YEP liquid media (peptone 10 g/L, yeast extract 10 g/L, beef extract 5 g/L, pH 7.0), and the culture was kept overnight at 28° C. 220 r/min. 2 ml of the bacteria that had been kept overnight was transferred into 50 mL of YEP liquid media, and cultured at 28° C. 220 r/min until the OD600 reached about 0.5. The cells were then incubated on ice for 30 minutes, and centrifuged at 4° C. 5000 rpm for 5 minutes. The supernatant was then abandoned. 20 ml of 50 mmol/L CaCl₂ was added to resuspend the cells. The cells were again centrifuged at 4° C. 5000 rpm for 5 minutes, and the supernatant was abandoned. 2 ml of 50 mmol/L CaCl₂ was added to resuspend the cells, and the cells were then aliquoted to 200 μl each, and stored at −80° C.

2. Transforming Agrobacterium tumefaciens

20 ng of purified plasmid pBIBS was added to 200 μl of Agrobacterium tumefaciens competent cells. After mixing up, the cells were incubated on ice for 5 minutes, and transferred to liquid nitrogen to freeze for 8 minutes. They were then immediately put at 37° C. to incubate for 5 minutes. The cells were then added into 800 μl of YEP liquid media, cultured at 28° C. 220 r/min for 4-5 hours, and then transferred onto the surface of the YEP solid media containing 50 mg/L of kanamycin. The cells should be evenly spread onto the whole plate. The cells were then cultured at 28° C. for 1-2 days.

3. Selecting and Identifying the Transformed Agrobacterium tumefaciens

5 ml of the YEP liquid media containing 50 mg/L of kanamycin were used to select those colonies carrying resistance. The cells were cultured at 28° C. 220 r/min for 1 day, centrifuged, and the plasmids were extracted and digested with restriction enzymes. The size of the fragment are detected by electrophoresis.

### Example 3

Pilot Experiments on the Antibiotics Sensitivity of Plant Cells

Gingseng callus cells (China Academy of Science, Shanghai Institute of Plant Physiology and Ecology, Fenglin Road 300, Shanghai, China, 200032) were cultured in the following four kinds of media. 1.5 gram of gingseng callus cells for solid culture were added into every 25 ml of the media.

1. The group for the resistance against ampicillin: 60 μl, 120 μl, 180 μl, 240 μl, 360 μl, or 500 μl of the 50 mg/ml ampicillin respectively were added into 25 ml of 67V solid media. The said 67V solid media contains in each liter of volumn the following contents: NaH₂PO₄.2H₂O 195 mg, Na₂HPO₄.12H₂O 50 mg, KCl 200 mg, MgSO₄.7H₂O 250 mg, (NH₄)₂SO₄ 100 mg, KNO₃ 800 mg, CaCl₂.2H₂O 200 mg, Na₂MoO₄.2H₂O 0.25 mg, CuSO₄.5H₂O 0.25 mg, MnSO₄.H₂O 4.00 mg, ZnSO₄.7H₂O 5.95 mg, CoCl₂.6H₂O 4.83 mg, H₃BO₃ 5.00 mg, KI 0.05 mg, hydrochloric thiamin 0.5 mg, hydrochloric pyridoxine 0.5 mg, nicotinic acid 1.25 mg, FeSO₄.7H₂O 13.9 mg, Na₂EDTA 18.5 mg, inositol 100 mg, sucrose 30 g, whey protein 1 g, agar powder 8 g, pH 5.8.

2. The group for the resistance against kanamycin: 5 μl, 10 μl, 25 μl, 50 μl, 75 μl, 100 μl or 150 μl of 50 mg/ml kanamycin respectively were added into 25 ml of 67V solid media.

3. The group for the resistance against G418: 5 μl, 10 μl, 25 μl, 50 μl, 75 μl, 100 μl, or 150 μl of 50 mg/ml G418 respectively were added into 25 ml of 67V solid media.

4. Control group: 25 ml of 67V solid media.

Growth within one month was compared: Between the group 1 which had different concentrations of antibiotics and the group 4, no significant difference was observed. Ginseng cells grew from 1.5 g to 6-7 g, showing that ginseng cells can grow normally at high concentration of ampicillin. Ampicillin therefore can be used in the selection process to suppress the growth of Agrobacterium tumefaciens, while in the meantime the growth of ginseng callus cells were not affected. Both group 2 and group 3 showed that low concentration of antibiotics suppressed the growth of ginseng cells, while high concentrations killed the ginseng cells. Ginseng cells were more sensitive to G418 than to kanamycin under the same concentration, showing that that G418 was more suitable to be used in the selection of transgenic ginseng cells. On the fourth day of culture, some of the cells in 150 μl of G418 started to die, while on the tenth day, almost all the cells were dead. On the twelfth day of culture, some of the cells in 25 μl of G418 started to die, while on the thirtieth day, almost all the cells were dead. The G418 concentration of 25 μl (50 mg/L) seemed to be suitable for the selection of transgenic ginseng cells.

### Example 4

Transforming Plant Cells with Agrobacterium tumefaciens

1. Preculture of Agrobacterium tumefaciens

Single colony of Agrobacterium tumefaciens was inoculated into 3 ml of YEP liquid media with antibiotics, and the bacteria were cultured at 28° C. 220 r/min until the OD600 reached around 0.9. 50 μl of the bacteria was put into 3 ml of AB media, and cultured at 28° C. 220 r/min until the OD600 reached around 0.9. The cells were then centrifuged at 4° C. 5000 rpm for 10 minutes. The pellet was the suspended in 50 ml of AB preinduction media (AB-AS), and cultured at 28° C. 220 r/min for 12-15 hours. The cells were then centrifuged at 4° C. 5000 rpm for 10 minutes, and resuspended in 20 ml of 67V-AS media.

2. Coculture of Agrobacterium tumefaciens and Plant Cells

The bacteria solution that had been precultured were poured into the flask which contained the ginseng callus cells, shaken well, and let stand for 15-20 minutes. The bacteria solution was then abandoned, and the plant cells were transferred into 67V-AS coculture solid media (which contained AS (acetosyringone) for a final concentration of 100 μM/L), cultured in dark at 25° C. for 48-72 hours. The coculture was transferred into a flask, and washed three times with the 67V media, once with 500 mg/L ampicillin, and then transferred into 67V solid media. The cells were cultured in dark at 25° C. for a week.

### Example 5

Selecting and Identifying the Transgenic Plant Cells

1. Selection by Resistance

The co-cultured cells were transferred onto the 67V solid media containing 300 mg/L of ampicillin and 50 mg/L of G418. After 4 weeks, the callus that bore resistance started to appear. The cells were passed once every two weeks. After 3 months, the media was changed into ampicillin-free 67V solid media containing 35 ml/L of G418, and the selection continued.

2. Identification of the Selected Cells

1) Detection of the Integration of Foreign Genes by PCR

50-200 mg of the cells that had been selected by resistance and cells of the negative control respectively were ground into powder after being frozen in liquid nitrogen. 900 μl of extraction buffer (100 mmol/L Tris.Cl (pH8.0), 50 mmol/L EDTA (pH8.0), 500 mmol/L NaCl, 10 mmol/L α-mercaptoethanol) was added, and mixed well. After adding 100 μl of 10% SDS, the cells were mixed up and incubated at 65° C. for 15 minutes. 160 μl of 5 mol/L potassium acetate were added, and the mixture obtained was mixed up and incubated on ice for 30 minutes. The mixture was then centrifuged at 4° C. 12000 rpm for 15 minutes. The supernatant was removed, into which equal volumn of chloroform/isopentanol was added. The mixture was then centrifuged at 4° C. 8000 rpm for 10 minutes. The supernatant was again removed, into which ⅔ volumn of pre-cooled isopropanol was added. The mixture was again mixed up and let stand for 30 minutes. After 10 minutes of centrifugation at 4° C. 8000 rpm, the supernatant was abandoned. The pellet was then washed with 80% ethanol and dried. 200 μl of TE buffer containing RNase was added to dissolve the precipitate, and the solution was incubated at 37° C. for 1 hour. Equal volumn of chloroform/isopropanol was added, and the mixture was centrifuged at 4° C. 8000 rpm for 10 minutes. The supernatant was removed, into which ⅔ volumn of pre-cooled isopropanol was added. The mixture was then mixed up and let stand for 30 minutes. After centrifugation at 4° C. 8000 rpm for 10 minutes, the supernatant was abandoned, and the pellet was washed with 80% ethanol, and dried out. 100 μl of TE (10 mmol/L Tris.Cl, pH 8.0, 1 mmol/L EDTA, pH8.0) was added to dissolve the DNA. Chromosomal DNA of both the selected cells carrying resistance and cells of the negative control were obtained. 1 μl of each was taken to be used as the template for PCR amplification. The PCR product of the selected cells carrying resistance was an amplified fragment of about 700 bp, while no specific amplified fragment was seen for the PCR amplification product of cells of the negative control.

2) Detection of the Expression of the Foreign Gene by ELISA

0.5 g of the selected cells carrying resistance and 0.5 g of cells of the negative control were ground into powder after being frozen in liquid nitrogen. 0.5 ml of extraction buffer (50 mmol/L Tris.Cl, 0.029% NaN₃ (pH9.5)) was added to the powder, and the solution was mixed up and extracted at 4° C. overnight. After centrifuged at 14000 rpm for 5 minutes, 50 μl supernatants of each were taken respectively, and expression of the antigen was detected using the HBsAg ELISA Detection Kit (commercially available from Hua Mei Biotechnology Company, San Shan Road 007, Opening Region of High and New Technological Industry, Luo Yang, 471003). Expression of the antigen was detected in the selected cells carrying resistance, while no expression of the antigen was detected in cells of the negative control.

We claim:

1. A construct comprising the HBsAg gene, in which a promoter that enables the highly-efficient expression of the gene is assembled at the 5′ end of the HBsAg gene, and a terminator that can enhance the expression of the gene is assembled at the 3′ end of the HBsAg gene.

2. The construct according to claim 1, wherein the promoter is CaMV 35S promoter.

3. The construct according to claim 1 wherein the terminator is nos terminator.

4. The construct according to claim 2 wherein the terminator is nos terminator.

5. The construct according to claim 1, further comprising a selective marker.

6. The construct according to claim 5, wherein the selective marker is NPT II.

7. A vector comprising the construct according to claim 1.

8. The vector according to claim 7 wherein the promoter is CaMV 35S promoter, and the said terminator is nos terminator.

9. The vector according to claim 7 further comprising a selective marker.

10. The vector according to claim 7, wherein the vector is selected from pBIBSa or pBIBSb.

11. An Agrobacterium tumefaciens comprising the vector of claim 7.

12. The Agrobacterium tumefaciens according to claim 11, which is Agrobacterium tumefaciens LBA4404.

13. A transgenic plant cell line comprising one selected from the group consisting of a construct, a vector comprising the construct, or a Agrobacterium tumefaciens comprising the construct, the construct comprising the HBsAg gene, in which a promoter that enables the highly-efficient expression of the gene is assembled at the 5′ end of the HBsAg gene, and a terminator that can enhance the expression of the gene is assembled at the 3′ end of the HBsAg gene, and wherein the cell line is ginseng cell line.

14. The transgenic plant cell line according to claim 13 wherein the cell line is of the ginseng callus.

15. A method for preparing a transgenic plant cell line comprising a construct or a vector comprising the HBsAg gene, comprising:
1) introducing into the plant cells a construct or vector comprising the HBsAg gene, in which a promoter that enables the highly-efficient expression of the gene is assembled at the 5′ end of the HBsAg gene, and a terminator which can enhance the expression of the gene is assembled at the 3′ end of the HBsAg gene; and
2) enabling the plant cells to express the HBsAg proteins.

16. The method according to claim 15, in which the the construct or vector is introduced into the plant cells by means selected from the group consisting of Agrobacterium tumefaciens infection, gene gun method, pollen introduction, virus-mediated method, PEG-mediated method, induction through electric shock, microinjection, laser transformation, ultra-sound transformation, and liposome transformation.

17. The method according to claim 16, the introduction means is by Agrobacterium tumefaciens infection.

18. The method according to claim 16, in which the construct or vector is introduced into the plant cells by co-culturing the Agrobacterium tumefaciens comprising the construct or vector and the suspension of the plant cells.

19. The method according to claim 17, in which during the process of Agrobacterium tumefaciens infection, a phenolic compound selected from the group consisting of catechol, gallic acid, pyrogallic acid, protocatechuic acid, vanillin, acetosyringone, and hydroxyl acetosyringone, is used to induce the activation of genes in the Vir region of Agrobacterium tumefaciens so as to enhance the transformation efficiency of Agrobacterium tumefaciens.

20. The method according to claim 19 wherein the phenolic compound is acetosyringone (AS).
